(19) United States
(12) Patent Application Publication
(10) Pub. No.: US 2008/0046073 A1
(43) Pub. Date: Feb. 21, 2008

(54) Non-Coagulative Vascular Shunt

(76) Inventor: H. Donel Elshire, San Diego, CA (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 11/839,922

(22) Filed: Aug. 16, 2007

Related U.S. Application Data
(60) Provisional application No. 60/838,533, filed on Aug. 16, 2006.

(51) Int. Cl. A61F 2/06

(52) U.S. Cl. 623/1.36; 604/8; 606/155

(57) ABSTRACT

An implantable shunt for the repair of a blood vessel, which is capable of remaining in place temporarily or for extended periods, which comprises a hollow tube, and flanges positioned at regular intervals along the tube's length, ending at both ends. The shunt is made of non-coagulative material. In operation, damaged vessels are severed and bridged by this implantable shunt, which is secured in place inside a vessel by applying ligatures to flanges on its end.

### RELATED APPLICATIONS

This is application claims the benefit of Provisional Application Ser. No. 60/838, 533, filed on Aug. 16, 2006. Application No. 60/838, 533 is hereby incorporated herein by reference.

### BACKGROUND OF INVENTION

1. Field of the Invention

The current invention relates to a vascular shunt, for the repair of an injured blood vessel, made of non-coagulative materials, such as polytetrafluoroethylene (PTFE). The shunt is capable of remaining in place in an injured blood vessel temporarily or for extended periods, in excess of two days, until adequate surgical facilities and capabilities are available for definitive repair of the vessel.

2. Description of Related Art

Vascular shunts are narrow, tubular devices that are used in surgical procedures for diverting blood flow through damaged vascular tissue. During operations, clamping off of major organs, such as the bowels, kidneys, liver, spleen, and body extremities for extended periods of time can lead to permanent damage or loss of the organ because of the lack of adequate blood flow. For example, damage to a kidney due to lack of blood flow may lead to the necessity of the patient to undergo dialysis treatment for the remainder of his life. Similarly, following an injury to an artery supplying an extremity, maintenance of an adequate blood supply to the extremity is often critical to avoid loss of the extremity.

A number of vascular shunt designs are currently available. They are designed for temporary replacement of a blood vessel, which lasts only hours. For example, a vascular shunt for use in surgical by-pass operations has been disclosed in U.S. Pat. No. 4,712,551 issued Dec. 15, 1987 to Rayhanabad. Additionally, polytetrafluoroethylene vascular grafts have been disclosed that when inserted provide long-term or permanent blood flow. Lentz, et al (U.S. Pat. Applications U.S. 2003/0004559 and US 2004/0193242) describe an implantable microporous vascular graft made of polytetrafluoroethylene.

Under circumstances where surgical procedures are not possible immediately after injury, for example in battlefield conditions, vascular shunts are inserted as a temporary measure to maintain perfusion of the organ. Most shunts are made of plastic or silicone rubber and occasionally contain a spring and tapered ends. Various sizes exist, depending upon the need for vessel caliber. They are usually secured at both ends with temporary suture material, umbilical tape, rubber bands or even small balloons.

These currently utilized devices, however, are designed to provide blood flow for a relatively short period of time, typically hours. Oftentimes vascular repair is not possible for a much longer period, such as in battlefield conditions or when individuals who are injured away from competent medical facilities. In these cases, shunts capable of remaining in place for an extended period until definitive surgical repair is available is critical.

### SUMMARY OF THE INVENTION

The inventive subject matter comprises a vascular shunt used for temporarily enabling blood flow to organs and extremities supplied by a damaged blood vessel. The inventive subject matter also comprises an easily implantable shunt, which may be used for extended periods of time, such as two or more days. The shunt is comprised of a hollow tube with inner and outer diameters of various dimensions to accommodate the damaged blood vessel.

The inventive subject matter further comprises a vascular shunt made of non-coagulative materials, that is designed for temporarily bridging a region of an injured blood vessel and for maintenance of blood supply to tissue until a more definitive repair is available. The non-coagulative material can be impregnated with drugs such as antibiotics, heparin, or other reinforcing structural materials.

The inventive subject matter also comprises a temporary vascular shunt, made from non-coagulative materials, that contains flanges embedded at regular intervals that are strong enough to secure ligatures.

The inventive subject matter further comprises a temporary vascular shunt that may be custom fit for each patient depending on the extent of damages to the blood vessel.

A further aspect of the inventive subject matter is a method of restoring blood flow to organs or extremities supplied by an injured blood vessel by shunting of the blood using an extended use vascular shunt.

### BRIEF DESCRIPTION OF THE DRAWINGS

FIG. 1. Lateral view of vascular shunt design.

FIG. 2. Cross-sectional view of vascular shunt without flange.

FIG. 3. Cross-sectional view of vascular shunt showing flange.

FIG. 4. Illustration of method of securing vascular shunt to blood vessel wall.

### DESCRIPTION OF THE PREFERRED EMBODIMENTS

The inventive subject matter comprises a shunt for the repair of a blood vessel, which is capable of remaining in place temporarily or for extended periods, such as in excess of 2 days. It is also possible, if medical conditions warrant it, that the shunt could remain in place permanently, thus obviating the further need of additional repair. An important aspect of the invention is that the shunt is made from materials that are strong and resistant to wear, with high chemical resistance and is resistant to platelet attachment or has characteristics that are non-permissive to blood clot formation. These properties enable the shunt to function safely inside a patient for an extended period. Materials commonly used in permanent vascular grafts may also be used to make the shunt. If the shunt is to be left in place for long periods or even permanently, the shunt may be stabilized by tissue growth around the shunt.

However, unlike a permanent vascular graft, the shunt of the current invention is implanted via a fairly simple procedure. Rather than time-consuming suturing, the shunt may be secured to the damaged blood vessel by ligatures. A ligature is a surgical thread, wire or cord for closing vessels or tying off ducts. To prevent deformation or collapse of the vessel from the circumferential force of ligature, the temporary shunt is reinforced at both ends with flanges.

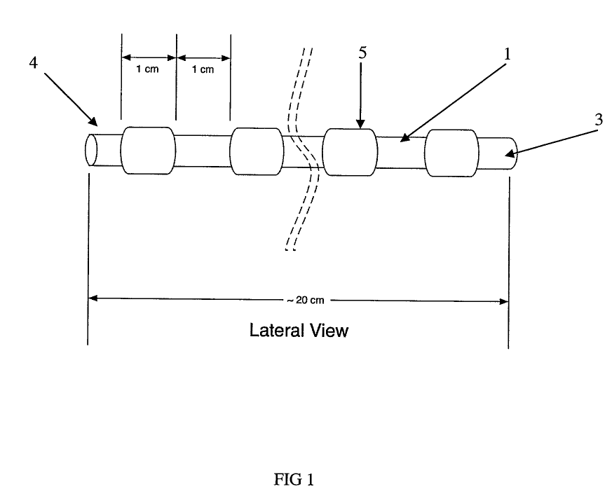

An illustrative example and preferred embodiment of the invention at FIG. 1, is a vascular shunt comprised of a hollow tube (1), with a proximal end (4) and a distal end (3) and is made of a non-coagulative material, such as expanded polytetrafluoroethylene (PTFE). However, the shunt can be made of any of a number of materials with similar properties as long as the material is not only resistant to platelet attachment but is also resistant to weathering, has low friction, high chemical resistance and broad temperature capability. In the preferred embodiment, a flange (5) is embedded at regular intervals, such as every other 1 cm, along the length of the tube (1). These flanges mark the length of each piece of shunt. In a preferred embodiment, the flange is made of a rigid material that will not collapse under the circumferential pressure of a ligature, such as heavy plastic. The flange may also be coated with a non-coagulative material, such as expanded PTFE-coated silicone plastic

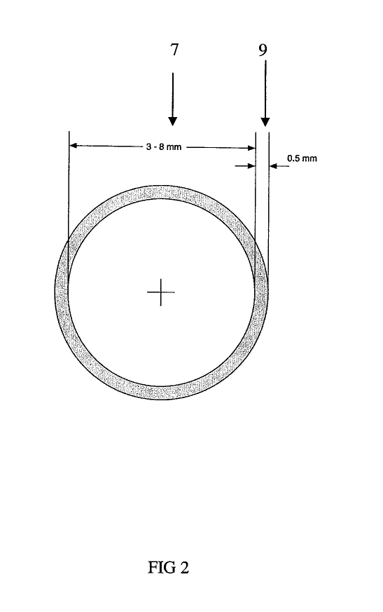

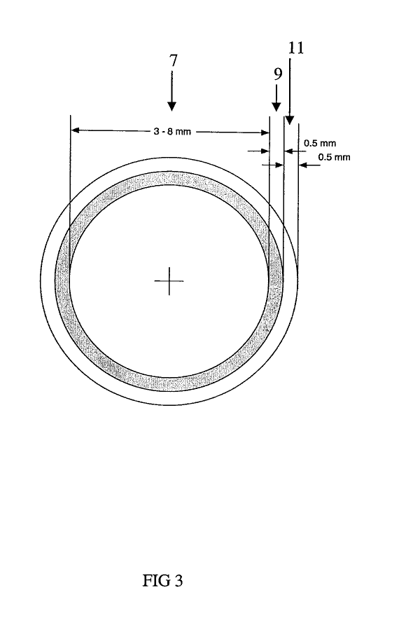

Referring to FIG. 2, the tube has an inside lumen diameter (7) and a thickness (9). In the preferred embodiment the tube thickness (9) is 0.5 mm or more. The inside lumen diameter (7) is variable to the blood vessel that the shunt will bridge. However, in the preferred embodiment, the inside lumen diameter (7) is 3 to 8 mm. Referring to FIG. 3, the flange, which is indicated as (5) in FIG. 1, is 0.5 mm thick (11). The preferred length of the shunt is 20 cm, although devices of other lengths are contemplated depending on the need. The contemplated invention may be stored either in pre-cut lengths or on sterile spools. Additionally, the device may be disposed of after use or sterilized by autoclaving or other sterilization methods for repeated use.

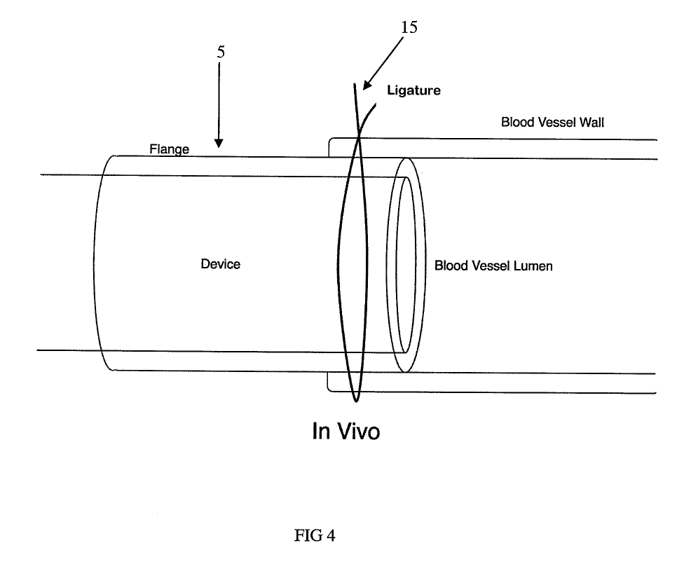

To permit blood flow through the injured blood vessel to the organ or extremity supplied by the blood vessel, the vessel is severed or an incision is made so that one end of the shunt may be inserted coaxially into a severed or cut end of the blood vessel. The opposite end of the shunt is similarly inserted into the other severed or cut end of the vessel, thus bridging the damaged blood vessel. Referring to FIG. 4, the shunt is held in place in the blood vessel lumen, with a seal afforded by a flange (5), by tying a ligature (15) around the vessel at the flange. Typically, 1 to 2 cm of the proximal and distal ends of the shunt is placed into the vessel lumen in order to secure it in place and to prevent inadvertent slippage or displacement.

Having described the invention, one of skill in the art will appreciate in the appended claims that many modifications and variations of the present invention are possible in light of the above teachings. It is, therefore, to be understood that, within the scope of the appended claims, the invention may be practiced otherwise than as specifically described.

What is claimed is:

1. A vascular shunt comprising a hollow tube having a proximal end and a distal end, an inside lumen diameter, and flanges positioned at regular intervals along the length of said tube ending at said proximal and distal ends, wherein said tube and flanges are made of a non-coagulative material.

2. The vascular shunt of claim 1, wherein said non-coagulative material is polytetrafluoroethylene (PTFE).

3. The vascular shunt of claim 1, wherein said non-coagulative material is a hydrophilic plastic polymer.

4. The vascular shunt of claim 1, wherein said non-coagulative material contains fillers selected from the group consisting of glass fibers, carbon, graphite molybdenum disulphide, and bronze.

5. The vascular shunt of claim 1, wherein said flanges are placed every other cm along the length of said tube.

6. The vascular shunt of claim 1, wherein said shunt is 20 cm long.

7. The vascular shunt of claim 1, wherein said inside lumen diameter is 3.0 to 8.0 mm.

8. The vascular shunt of claim 1, wherein said tube is at least 0.5 mm thick.

9. The vascular shunt of claim 1, wherein said flange is at least 0.5 mm thick.

10. The vascular shunt of claim 1, wherein said flange is a circular ring formed on the outside of said tube.

11. A vascular shunt comprising a hollow tube having a proximal end and a distal end, an inside lumen diameter, and flanges positioned at regular intervals along the length of said tube ending at said proximal and distal ends, wherein said tube is made of a non-coagulative material, and said flange is made of a rigid material and coated with a non-coagulative material.

12. The vascular shunt of claim 11, wherein said rigid material is a heavy plastic.

13. The vascular shunt of claim 12, wherein said plastic is silicone plastic.

14. The vascular shunt of claim 11, wherein said non-coagulative material is expanded PTFE.

15. A method of restoring blood flow to organs or extremities supplied by a damaged blood vessel comprising the steps:
a. severing or making incisions in the damaged blood vessel to create two openings, each large enough to receive an end of a vascular shunt, having a flange on said end;
b. inserting 1 to 2 cm of an end of said shunt into each of said vessel openings, such that said flange is inserted into said vessel; and
c. securing each end of the shunt at said flange with a ligature.

16. A method of claim 15, wherein prior to insertion, said vascular shunt is cut to desired length from a spool of said shunt.
